(19) United States
(12) Patent Application Publication
(10) Pub. No.: US 2023/0089557 A1
(43) Pub. Date: Mar. 23, 2023

(54) METHODS OF TREATING DLBCL USING BTK INHIBITORS AND COMBINATIONS THEREOF

(71) Applicant: BEIGENE SWITZERLAND GMBH, Basel (CH)

(72) Inventors: Yiling YU, Beijing (CN); Yang LIU, Beijing (CN); Weige WANG, Beijing (CN); Hui YAO, Beijing (CN); Xinfeng HOU, Beijing (CN); Haiyi GUO, Beijing (CN); Zhirong SHEN, Beijing (CN); Lai WANG, Beijing (CN)

(21) Appl. No.: 17/802,111

(22) PCT Filed: Feb. 25, 2021
(86) PCT No.: PCT/CN2021/077935

§ 371 Date: Aug. 24, 2022

(30) Foreign Application Priority Data
Feb. 27, 2020 (CN) PCT/CN2020/077027

(51) Int. Cl. A61K 31/519; A61P 35/00; C07K 16/28

(52) U.S. Cl. CPC A61K 31/519; A61P 35/00; C07K 16/2887

(57) ABSTRACT

It provides methods of treating no-GCB DLBCL with (S)-7-(1-acryloylpiperidin-4-yl)-2-(4-phenoxyphenyl)-4, 5, 6, 7-tetrahydropyrazolo-[1, 5-a] pyrimidine-3-carboxamide or a pharmaceutically acceptable salt thereof. It provides methods of treating non-GCB DLBCL in a subject, comprising administering to the subject in need thereof (S)-7-(1-acryloylpiperidin-4-yl)-2-(4-phenoxyphenyl)-4, 5, 6, 7-tetrahydropyrazolo-[1, 5-a] pyrimidine-3-carboxamide or a pharmaceutically acceptable salt thereof, in combination with an anti-CD20 antibody.

### FIELD OF THE DISCLOSURE

Disclosed herein are methods of treating non-GCB DLBCL in a subject, comprising administering to the subject in need thereof (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof. Disclosed herein are also methods of treating non-GCB DLBCL in a subject, comprising administering to the subject in need thereof (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof, in combination with an anti-CD20 antibody.

### BACKGROUND OF THE DISCLOSURE

Diffuse large B-cell lymphoma (DLBCL), the most common subtype of non-Hodgkin's lymphoma, is clinically heterogeneous: 40% of patients respond well to current therapy and have prolonged survival, whereas the remainder succumb to the disease. (Alizadeh et al., Nature 2000; 403(6769):503-11). DLBCL can be divided into prognostically significant subgroups with germinal center B-cell-like (GCB), activated B-cell-like (ABC), expression profiles (Alizadeh supra). The GCB group has a better survival rate than the ABC group, but both indications are associated with poor clinical outcomes. The use of molecular profiling to predict survival for DLBCL has seen previous use (Rosenwald et al., N Engl J Med. 2002; 346: 1937-1947). In 2016, the World Health Organization (WHO) revised their classification of lymphoid neoplasms to account for the major advances in lymphoma biology since 2008 (Swerdlow et al., Blood. 2016; 127(20):2375-2390). The WHO classification emphasized gene expression and alterations of clinical importance, such as changes in the MYC, BCL2, and/or BCL6 oncogenes. More than 80% of patients with the so-called “double-hit” lymphoma have translocations in MYC and BCL2 (Aukema et al., Blood. 2011; 117(8):2319-2331). This indicates a need for additional molecular markers other than MYC and BCL2 that could be used to classify patients prior to treatment or to indicate response to treatment.

Bruton's tyrosine kinase (BTK) inhibitors alone or in combination with CD20 antibody only showed modest activity in R/R non-GCB DLBCL. However, patients with CD79B mutations responded to ibrutinib frequently (5/9; 55%), especially those with concomitant myeloid differentiation primary response 88 (MYD88) mutations (4/5; 80%). A phase 3 PHONIX trial of ibrutinib plus R-CHOP failed to meet its primary endpoints for treating frontline non-GCB DLBCL patients, however, biomarker analysis showed improved event-free survival in BCL2/MYC co-expression population. And these benefits were only observed in patients younger than 65 which also suggested a relatively high toxicity may be caused by the combinational use of ibrutinib and R-CHOP. So, these clinical outcomes indicate that patient stratification and patient response based on molecular markers will be the key for future non-GCB DLBCL treatment and regimens with less toxicity should be further investigated.

### SUMMARY OF THE DISCLOSURE

WO2014/173289A disclosed a series of BTK inhibitors, particularly (S)-7-(1-acryloylpiperidin-4-yl)-2-(4-phenoxyphenyl)-4,5,6,7-tetra-hydropyrazolo[1,5-a]pyrimidine-3-carboxamide (hereinafter Compound 1). Compound 1 can be used for the treatment of cancers with aberrations in the B-cell receptor (BCR) and FcR signaling pathway in which BTK plays important roles and has demonstrated to have potent and irreversible inhibitory activities against BTK.

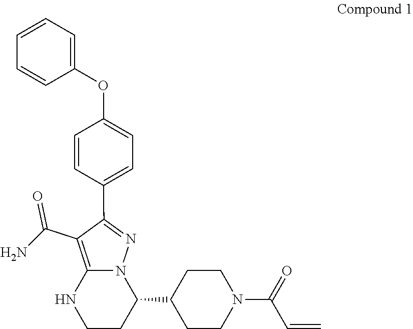 [SMILES: C=CC(=O)N1CCC([C@@H]2CCNc3c(C(N)=O)c(-c4ccc(Oc5ccccc5)cc4)nn32)CC1]

The present disclosure describes (S)-7-(1-acryloylpiperidin-4-yl)-2-(4-phenoxyphenyl)-4,5,6,7-tetra-hydropyrazolo[1,5-a]pyrimidine-3-carboxamide (Compound 1) or a pharmaceutically acceptable salt thereof alone, and a combination of Compound 1 with anti-CD20 antibody, showed sensitive response in some populations of non-GCB DLBCL or ABC DLBCL, particularly those with a specific gene high expression, including PAX5, PIM1, BCL2, FOXP1, BCL2, MYC, or BCL2/MYC.

The present disclosure also describes Compound 1 or a pharmaceutically acceptable salt thereof when administered alone, or administered as a combination of Compound 1 with anti-CD20 antibody, showed sensitive response in some populations of non-GCB DLBCL, particularly those with a specific gene mutations, including mutations of BCR or NOTCH1 pathways, such as CD79B mutations or NOTCH1 mutations.

The inventors of the present disclosure discovered that ABC or non-GCB DLBCL is more sensitive to Compound 1 than GCB subtype in in-vitro and in-vivo assays.

In clinical studies, the inventors discovered that PAX5 expression was significantly higher in Compound 1 monotherapy responders, and PIM1, BCL2, and FOXP1 expression was higher in Compound 1 combination therapy responders. In Compound 1 monotherapy, the inventors also discovered patients with NOTCH1 mutations had higher ORR. In both Compound 1 monotherapy and combination therapy of Compound 1 in combined with anti-CD20 antibody, the inventors also discovered that patients with MYC, BCL2, MYC and BCL2 double expressor DLBCL tended to have higher ORR and longer progression-free survival and overall survival, and patients with CD79B showed significantly higher ORR than patients without CD79B mutations.

In addition, the inventors of present disclosure discovered that EBV negative non-GCB DLBCL were more sensitive to Compound 1 than EBV positive non-GCB DLBCL.

In first aspect, disclosed herein is a method of treating non-GCB DLBCL or ABC DLBCL in a subject, comprising administering to the subject in need thereof a therapeutically effective amount of Compound 1 or a pharmaceutically acceptable salt thereof.

Also disclosed herein is a pharmaceutical composition for use in the treatment of non-GCB DLBCL or ABC DLBCL, comprising Compound 1 or a pharmaceutically acceptable salt thereof.

In one embodiment, the non-GCB DLBCL is a non-GCB DLBCL with a gene high expression of PAX5, BCL2, MYC, or BCL2/MYC.

In an embodiment, the non-GCB DLBCL is a non-GCB DLBCL with BCR pathway gene mutations or NOTCH1 pathways gene mutations. In a preferred embodiment, the non-GCB DLBCL is a non-GCB DLBCL with a CD79B mutation, or a NOTCH1 mutation

In second aspect, disclosed herein is a method of treating non-GCB DLBCL or ABC DLBCL in a subject, comprising administering to the subject in need thereof a therapeutically effective amount of Compound 1 or a pharmaceutically acceptable salt thereof, in combination with an anti-CD20 antibody.

In yet another aspect, disclosed herein Compound 1 or a pharmaceutically acceptable salt thereof, for use in the treatment of non-GCB DLBCL or ABC DLBCL, in combination with an anti-CD20 antibody.

The disclosure also provides for a use of a pharmaceutical combination in the manufacture of a medicament for use in the treatment of non-GCB DLBCL or ABC DLBCL, and the pharmaceutical combination comprising Compound 1 or a pharmaceutically acceptable salt thereof, and an anti-CD20 antibody.

Articles of manufacture, or “kits” comprising a first container, a second container and a package insert, wherein the first container comprises at least one dose of a medicament comprising Compound 1 or a pharmaceutically acceptable salt thereof, the second container comprises at least one dose of a medicament comprising an anti-CD20 antibody, and the package insert comprises instructions for treating non-GCB DLBCL in a subject using the medicaments is also included.

In one embodiment, the anti-CD20 antibody is selected from rituximab, ibritumomab tiuxetan, tositumomab, ofatumumab or obinutuzumab.

In one embodiment, the non-GCB DLBCL is a non-GCB DLBCL with a gene high expression of PIM1, BCL2, FOXP1, MYC, or BCL2/MYC.

In one embodiment, the non-GCB DLBCL is a non-GCB DLBCL with BCR pathway gene mutations or NOTCH1 pathways gene mutations. In a preferred embodiment, the non-GCB DLBCL is a non-GCB DLBCL with a CD79B mutation, or a NOTCH1 mutation.

In one embodiment of each of the above aspects, the non-GCB is a R/R non-GCB DLBCL.

In one embodiment of each of the above aspects, the non-GCB is a R/R ABC DLBCL.

In third aspect, disclosed herein is a method of increasing the response of a cancer patient for treatment with a BTK inhibitor, the method comprising:

a) administration of a BTK inhibitor to the patient;

b) assaying for gene expression of PAX5 in a cancer sample obtained from the patient;

c) comparing the gene expression of PAX5, CD79B mutation or NOTCH1 mutation in the cancer sample obtained from a patient treated with a BTK inhibitor with that of a cancer sample prior to BTK inhibitor administration;

d) wherein increased expression of PAX5, CD79B mutation or NOTCH1 mutation indicates that the patient will be sensitive to BTK inhibitor treatment.

In four aspect, disclosed herein provide a method of increasing the response of a cancer patient for treatment with a BTK inhibitor in combination with an anti-CD20 antibody, the method comprising:

a) administration of a BTK inhibitor in combination with an anti-CD20 antibody to the patient;

b) assaying for gene expression of any one of PIM1, BCL2, FOXP1, MYC or CD79B mutation in a cancer sample obtained from the patient;

c) comparing the gene expression of any one of PIM1, BCL2, FOXP1, MYC or CD79B mutation in the cancer sample obtained from a patient treated with a BTK inhibitor in combination with an anti-CD20 antibody with that of a cancer sample prior to BTK inhibitor in combination with an anti-CD20 antibody administration;

d) wherein increased expression of PIM1, BCL2, FOXP1, MYC or CD79B mutation indicates that the patient will be sensitive to BTK inhibitor treatment.

### BRIEF DESCRIPTION OF THE DRAWINGS

FIG. 1 showed ABC/non-GCB DLBCL is more sensitive to Compound 1 than GCB subtype.

DLBCL PDX models were classified into GCB and non-GCB subtypes and treated with compound 1 as described in methods. Tumor growth inhibition (TGI) of compound 1 in each mice was calculated. Each dot points represent the mean TGI of one model. P<0.05 (two tails t test).

FIG. 2 showed non-GCB DLBCL PDX models with CD79B mutation, or CD79B-ITAM domain (amino acid 193-210) mutation are more sensitive to Compound 1.

FIG. 2(A): non-GCB DLBCL PDX models were treated with Compound 1 and TGI of each model were calculated described in methods. The models were subdivided into CD79B wildtype (CD79B-WT) and CD79B mutation (CD79B-mut) groups. Each dot points represent the mean TGI of one model. P<0.05 (two tails t test).

FIG. 2(B): non-GCB DLBCL PDX models were treated with Compound 1 and TGI of each model were calculated described in methods. The models were subdivided into CD79B ITAM domain (amino acid 193-210) wildtype (CD79B ITAM-WT) and CD79B ITAM domain mutation (CD79B ITAM-mut) groups. Each dot points represent the mean TGI of one model. P<0.05 (two tails t test).

FIG. 3 showed EBV negative non-GCB DLBCL PDX models are more sensitive to Compound 1.

Non-GCB DLBCL PDX models were treated with Compound 1 and TGI of each mice were calculated as described in methods. The models were subdivided into Epstein-Barr virus (EBV) negative (EBV−) and EBV positive (EBV+) groups. Each dot points represent the mean TGI of one model. P<0.05 (two tails t test).

FIG. 4 showed 56 non-GCB DLBCL subtyped by the GEP method were subjected to mRNA analysis (total 95 genes).

FIG. 4(A) showed PAX5 was enriched in responders in Compound 1 monotherapy schedules 1 and 2.

FIG. 4(B) showed PIM1, FOXP1 and BCL2 were enriched in responders to Compound 1 in combination with CD20 antibody via schedules 3 and 4.

FIG. 5 showed best observed response (BOR), progression free survival (PFS) and overall survival (OS) in 28 BCL2-high and 28 BCL2-low patients (median expression value of BCL2 was used as the threshold for defining BCL2-high or BCL2-low).

FIG. 6 showed best observed response (BOR), progression free survival (PFS) and overall survival (OS) in 28 MYC-high and 28 MYC-low patients (median expression value of MYC was used as the threshold for defining MYC-high or MYC-low).

FIG. 7 showed best observed response (BOR), progression free survival (PFS) and overall survival (OS) in 18 DE patients and 38 non-DE patients (Median expression value of BCL2/MYC were used as threshold for defining DE or non-DE).

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The present disclosure provides a method of treating non-GCB DLBCL or ABC DLBCL in a subject, comprising administering to the subject in need thereof Compound 1 or a pharmaceutically acceptable salt thereof.

The present disclosure also provides a method of treating non-GCB DLBCL or ABC DLBCL in a subject, comprising administering to the subject in need thereof Compound 1 or a pharmaceutically acceptable salt thereof, in combination with an anti-CD20 antibody.

The present disclosure also provides a method of increasing the response of a cancer patient for treatment with a BTK inhibitor, the method comprising:

a) administration of a BTK inhibitor to the patient;

b) assaying for gene expression of PAX5 in a cancer sample obtained from the patient;

c) comparing the gene expression of PAX5 in the cancer sample obtained from a patient treated with a BTK inhibitor with that of a cancer sample prior to BTK inhibitor administration;

d) wherein increased expression of PAX5 indicates that the patient will be sensitive to BTK inhibitor treatment.

The present disclosure also provides a method of increasing the response of a cancer patient for treatment with a BTK inhibitor in combination with an anti-CD20 antibody, the method comprising:

a) administration of a BTK inhibitor in combination with an anti-CD20 antibody to the patient;

b) assaying for gene expression of any one of PIM1, BCL2, FOXP1 or MYC in a cancer sample obtained from the patient;

c) comparing the gene expression of any one of PIM1, BCL2, FOXP1 or MYC in the cancer sample obtained from a patient treated with a BTK inhibitor in combination with an anti-CD20 antibody with that of a cancer sample prior to BTK inhibitor in combination with an anti-CD20 antibody administration;

d) wherein increased expression of PIM1, BCL2, FOXP1 or MYC indicates that the patient will be sensitive to BTK inhibitor treatment.

### Anti-CD20 Antibody

The “anti-CD20 antibody” includes, but not limited to, rituximab, ibritumomab tiuxetan, tositumomab, ofatumumab or obinutuzumab.

### Methods of Treatment

In one aspect, the present disclosure provides a method of treating non-GCB DLBCL or ABC DLBCL in a subject.

In certain aspects, the method comprises administering to the subject in need thereof Compound 1 or a pharmaceutically acceptable salt thereof.

In an embodiment, the non-GCB DLBCL is a non-GCB DLBCL with a gene high expression, selected from the group consisting of PAX5, BCL2, MYC, or BCL2/MYC.

In one embodiment, the non-GCB DLBCL is a non-GCB DLBCL with BCR pathway gene mutations or NOTCH1 pathways gene mutations. In a preferred embodiment, the non-GCB DLBCL is a non-GCB DLBCL with a CD79B mutation, or a NOTCH1 mutation.

In yet another aspect, disclosed herein is Compound 1 or a pharmaceutically acceptable salt thereof, for use in the treatment of non-GCB DLBCL or ABC DLBCL, in combination with an anti-CD20 antibody.

In an embodiment, the non-GCB DLBCL is a non-GCB DLBCL with a gene high expression, selected from the group consisting of PIM1, BCL2, FOXP1, MYC, or BCL2/MYC.

In an embodiment, the non-GCB DLBCL is a non-GCB DLBCL with BCR pathway gene mutations or NOTCH1 pathways gene mutations. In a preferred embodiment, the non-GCB DLBCL is a non-GCB DLBCL with a CD79B mutation, or a NOTCH1 mutation.

Compound 1 can be administered by any suitable means, including oral, parenteral, intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound 1 would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the prevention or treatment of disease, the appropriate dosage of Compound 1 and anti-CD20 antibody in a monotherapy or in a combination therapy will depend on the type of disease to be treated, the severity and course of the disease.

In some embodiments, Compound 1 is orally administrated at dose of 40 mg-640 mg per day. In some embodiments, Compound 1 is orally administrated at dose of 100 mg-400 mg per day. In some embodiments, Compound 1 is orally administrated at dose of 320 mg QD or 160 mg BID.

In some embodiments, the anti-CD20 antibody is obinutuzumab. In some embodiments, Compound 1 is orally administrated at a dose of 320 mg QD or 160 mg BID in 28-day cycles, and obinutuzumab is administrated by three loading doses of 1000 mg weekly followed by 1000 mg on day one of cycles 2-6.

In some embodiments, the anti-CD20 is rituximab. Compound 1 was administrated in combination with rituximab. In some embodiments, Compound 1 is orally administrated at a dose of 320 mg QD or 160 mg BID, rituximab is administered at 375 mg/m2 intravenously on Cycle 1 Days 1, 8, 15, 22, and on Day 1 of Cycles 4, 6, 8, 10, and Compound 1 is administered at least 30 minutes prior to the initiation of the rituximab infusion.

ABBREVIATIONS AND DEFINITIONS OF TERMS

| Abbreviation | Term |
|---|---|
| ABC-DLBCL | activated B cell-like DLBCL |
| BID | twice daily |
| BOR | best overall response |
| CLL | chronic lymphocytic leukemia |
| CR | complete response |
| DE | Double Expressor |
| DLBCL | diffuse large B-cell lymphoma |
| DOR | duration of response |
| GCB | germinal center B-cell-like |
| NGS | next generation sequencing |
| ORR | overall response rate |
| OS | overall survival |
| PD | progressive disease |
| PFS | progression-free survival |
| PR | partial response |
| PT | preferred term |
| QD | once daily |
| R/R | relapsed/refractory |

### EXAMPLES

The present invention is further exemplified, but not limited to, by the following examples that illustrate the invention.

### Example 1 Comparison of DLBCL Cell of Origin (COO) to the Influence of Compound 1 Response Methods

DLBCL patient-derived xenografted (PDX) models were established in immunodeficient NCG (NOD-Prkdcem26Cd52Il2rgem26Cd22/NjuCrl) mice. These models were classified into GCB and non-GCB subgroups based on immunohistochemistry (IHC) staining using Hans algorithm (Hans, C. P., et al., Blood, 2004. 103 (1): p. 275-282). For efficacy studies, tumor tissues were inoculated subcutaneously into NCG mice. When tumors reached 100-200 mm³, mice were randomized into each group with 7-10 mice/group based on tumor volume. Compound 1 was formulated with 0.5% methyl cellulose as vehicle and treatment was started on the day of randomized grouping. Compound 1 was administrated p.o., 7.5 mg/(kg body weight), BID. Tumor volumes were measured twice weekly in two dimensions using a caliper, and the volume were expressed in mm³ using the formula:

V=0.5(a×b²) where a and b are the long and short diameters of the tumor, respectively.

Tumor growth inhibition (TGI) was calculated using the following formula:

% TGI=100×[1−(treatmentᵥₜ−treatmentᵥ₀)/(vehicleᵥₜ−Vehicleᵥ₀)]

Treatmentᵥₜ=tumor volume of treatment group at time t.

treatmentᵥ₀=tumor volume of treatment group at time of treatment started.

Vehicleᵥₜ=tumor volume of vehicle group at time t.

Vehicleᵥ₀=tumor volume of vehicle group at time of treatment started.

P value was calculated using two tails t test.

### Results

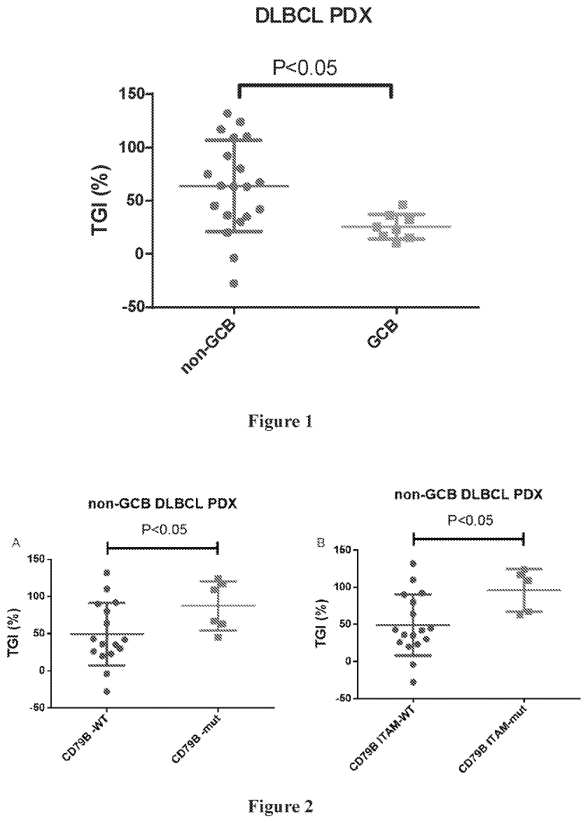

Compound 1 resulted in higher TGI in non-GCB DLBCL (TGI 63.60±42.92, mean±SD) models than that of GCB subtype (TGI 25.5±11.96, mean±SD), P<0.05 (two tails t test). See FIG. 1.

### Conclusion

DLBCL of non-GCB subtype were significantly more sensitive to Compound 1 than those of GCB subtype.

### Example 2 Comparison of CD79B and CD79B ITAM Domain Mutation to the Sensitivity of Compound 1 in DLBCL

### Methods

DLBCL patient-derived xenografted (PDX) models were established in immunodeficient NCG (NOD-Prkdcem26Cd52Il2rgem26Cd22/NjuCrl) mice. These models were classified into GCB and non-GCB subgroups based on immunohistochemistry (IHC) staining using Hans algorithm. Non-GCB DLBCL PDX models were classified into CD79B mutant versus CD79B wildtype groups; or CD79B ITAM domain mutant (amino acid 193-210) versus CD79B ITAM domain wildtype groups. For efficacy studies, tumor tissues were inoculated subcutaneously into NCG mice. When tumors reached 100˜200 mm³, mice were randomized into each group with 7˜10 mice/group based on tumor volume. Compound 1 was formulated with 0.5% methyl cellulose as vehicle and treatment was started on the day of randomized grouping. Compound 1 was administrated p.o., 7.5 mg/(kg body weight), BID. Tumor volumes were measured twice weekly in two dimensions using a caliper, and the volume were expressed in mm³ using the formula:

V=0.5(a×b²) where a and b are the long and short diameters of the tumor, respectively.

Tumor growth inhibition (TGI) was calculated using the following formula:

% TGI=100×[1−(treatmentᵥₜ−treatmentᵥ₀)/(vehicleᵥₜ−Vehicleᵥ₀)]

Treatmentᵥₜ=tumor volume of treatment group at time t.

treatmentᵥ₀=tumor volume of treatment group at time of treatment started.

Vehicleᵥₜ=tumor volume of vehicle group at time t.

Vehicleᵥ₀=tumor volume of vehicle group at time of treatment started.

P value was calculated using two tails t test.

### Results

Compound 1 resulted in higher TGI in CD79B mutation (TGI 87.5±33.14, mean±SD) or CD79B ITAM domain mutation (TGI 96.00±28.83, mean±SD) non-GCB DLBCL models than that of CD79B wildtype (TGI 49.44±42.36, mean±SD) or CD79B ITAM domain wildtype (TGI 49.1841.03, mean±SD) non-GCB DLBCL models, P<0.05 (two tails t test). See FIG. 2.

### Conclusion

Non-GCB DLBCL with CD79B gene mutation were more sensitive to Compound 1 than those with wildtype CD79B gene. Non-GCB DLBCL with CD79B ITAM domain mutation were more sensitive to compound 1 than those without CD79B ITAM domain mutation.

### Example 3 Comparison of EBV Infection Status to the Sensitivity of Compound 1 in DLBCL

### Methods

DLBCL patient-derived xenografted (PDX) models were established in immunodeficient NCG (NOD-Prkdcem26Cd52Il2rgem26Cd22/NjuCrl) mice. These models were classified into GCB and non-GCB subgroups based on immunohistochemistry (IHC) staining using Hans algorithm. Non-GCB DLBCL PDX models were classified into Epstein-Barr virus (EBV) negative and EBV positive subgroups. For efficacy studies, tumor tissues were inoculated subcutaneously into NCG mice. When tumors reached 100˜200 mm³, mice were randomized into each group with 7˜10 mice/group based on tumor volume. Compound 1 was formulated with 0.5% methyl cellulose as vehicle and treatment was started on the day of randomized grouping. Compound 1 was administrated p.o., 7.5 mg/(kg body weight), BID. Tumor volumes were measured twice weekly in two dimensions using a caliper, and the volume were expressed in mm³ using the formula:

V=0.5(a×b²) where a and b are the long and short diameters of the tumor, respectively.

Tumor growth inhibition (TGI) was calculated using the following formula:

% TGI=100×[1−(treatmentᵥₜ−treatmentᵥ₀)/(vehicleᵥₜ−Vehicleᵥ₀)]

Treatmentᵥₜ=tumor volume of treatment group at time t.

treatmentᵥ₀=tumor volume of treatment group at time of treatment started.

Vehicleᵥₜ=tumor volume of vehicle group at time t.

Vehicleᵥ₀=tumor volume of vehicle group at time of treatment started.

Average TGI of each model was used and represented as a dot point in the figure. P value was calculated using two tails t test to compare difference between groups.

### Results

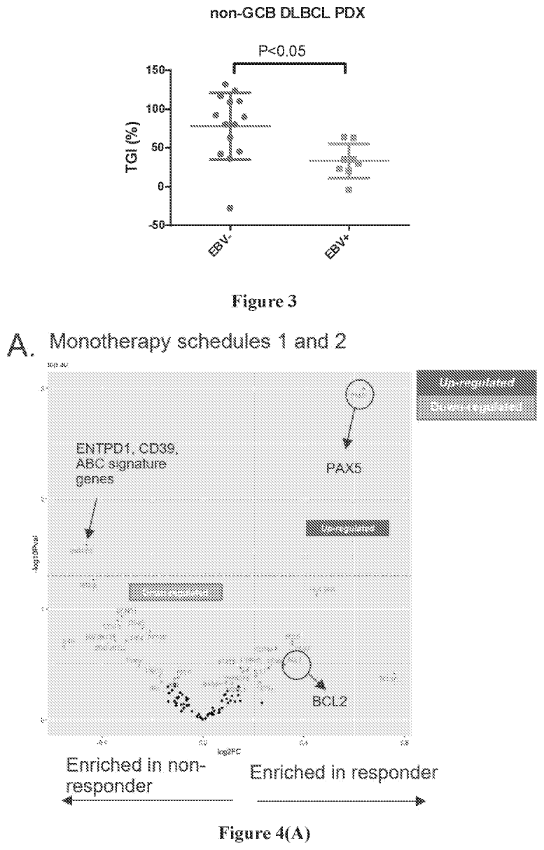

Compound 1 resulted higher TGI in EBV negative (TGI 78.00±43.41, mean±SD) non-GCB DLBCL models than that of EBV positive (TGI 33.25±22.41, mean±SD) models, P<0.05 (two tails t test). See FIG. 3.

### Conclusion

EBV negative non-GCB DLBCL were more sensitive to Compound 1 than EBV positive non-GCB DLBCL.

### Example 4 Treatment of Non-Germinal Center B-Cell Like Diffuse Large B-Cell Lymphomas (Non-GCB DLBCL) with BTK Inhibitor Treatment

A total of 121 patients with R/R non-GCB DLBCL were recruited in four Compound 1 treatment studies that were conducted at a similar time period. Two of the four studies were Compound 1 monotherapy (n=79) and two were Compound 1 combined with an anti-CD20 antibody therapy (n=42). Similar inclusion and exclusion criteria and response evaluation criteria were used across all studies. Fifty-six non-GCB patients were further subtyped by gene expression profiling (GEP) using the HTG EdgeSeq DLBCL Cell of Origin Assay. The expression of approximately 90 lymphoma-associated genes from the HTG GEP assay were analyzed by R package limma for correlation with response to Compound 1 treatment. Seventy-seven patient samples were tested by next-generation sequencing (NGS) with a panel of genes. Chi-square test was used to evaluate the correlation between mutations and the objective response rate (ORR).

### Example 4A: Treatment of Non-GCB DLBCL with BTK Inhibitor Monotherapy

### Methods

Monotherapy schedule 1: Compound 1 was administered orally every day at 160 mg twice daily (BID) in 28-day cycles in patients with R/R non-GCB DLBCL.

Monotherapy schedule 2: Compound 1 was administered orally every day in each cycle at 160 mg BID in 28-day cycles in patients with R/R non-GCB DLBCL.

79 patients with non-GCB DLBCL were subjected to Compound 1 monotherapies (Monotherapy Schedule 1 or 2).

### Results

The unadjusted ORR in non-GCB DLBCL was 31.6% and 29.3% for the two monotherapies. For patients who GEP-confirmed activated B-cell DLBCL classification, the ORR was 53.8% and 36%, respectively.

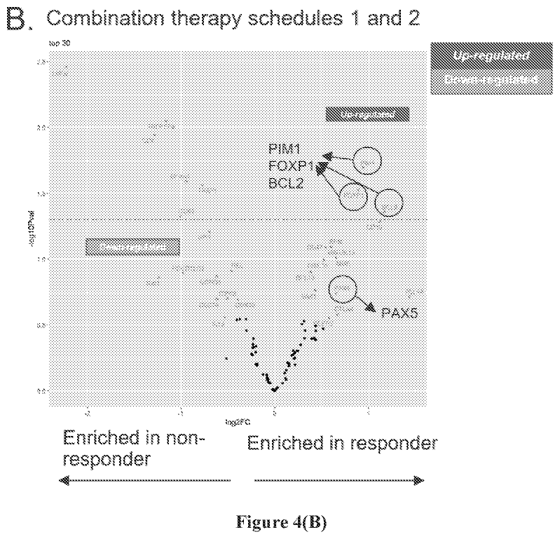

For the 44 non-GCB patients with HTG gene expression profiles, PAX5 expression was significantly higher in the two monotherapies responders. (See FIG. 4).

For the 44 patients with non-GCB DLBCL underwent panel sequencing, the mutations of B-cell receptor (BCR) pathway or NOTCH1 pathway related genes were identified to be correlated with better response. Patients with non-GCB DLBCL with CD79B mutations (n=17) showed higher ORR than other population (52.9% vs. 33.3%, p=0.2). Patients with NOTCH1 mutant (n=3) had higher ORR in the two monotherapies (100%/vs. 36.6%, p<0.05). See Table 1.

TABLE 1

| | Compound 1 Monotherapy | | |
|---|---|---|---|
| | 160 BID | 160 BID | |
| | (Schedule 1; N = 38) | (Schedule 2; N = 41) | Total (N = 79) |
| non-GCB | Response/Patient ORR | Response/Patients ORR | Response/Patients ORR |
| Overall Response | | | |
| Non-GCB DLBCL Patients | 12/38 31.6 | 12/41 29.3 | 24/79 30.4 |
| Patients with Sequence | 7/11 63.6 | 12/34 35.3 | 19/45 42.2 |
| Available | | | |
| Non-GCB DLBCL Patients | 6/10 60.0 | 12/34 35.3 | 18/44 40.9 |
| with Sequence Available | | | |
| ABC Subtype of | 7/13 53.8 | 9/25 36.0 | 16/38 42.1 |
| DLBCL Patients | | | |
| NOTCH1 | | | |
| NOTCH1_Mut | 1/1 100.0 | 2/2 100.0 | 3/3 100.0 |
| NOTCH1_WT | 5/9 55.6 | 10/32 31.3 | 15/41 36.6 |
| Difference in ORR | 44.4 (−45.95, | 68.8 (−0.40, | 63.4 (4.48, |
| (95% CI) [1] | 74.47) | 82.20) | 76.54) |
| Two-sided P-value | 0.3894 | 0.0484 | 0.0310 |
| MYD88/CD79B | | | |
| MYD88_L265P/CD79B_Mut | | 3/6 50.0 | 3/6 50.0 |
| Other | 6/1 0 60.0 | 9/28 32.1 | 15/38 39.5 |
| Difference in ORR | | 17.9 (−20.09, | 10.5 (−25.93, |
| (95% CI) [1] | | 54.50) | 46.49) |
| Two-sided P-value | | 0.4062 | 0.6260 |
| MYD88 mutation | | | |
| MYD88_L265P | 0/1 0.0 | 4/10 40.0 | 4/11 36.4 |
| MYD88_WT | 6/9 66.7 | 8/24 33.3 | 14/33 42.4 |
| Difference in ORR | −66.7 (−88.57, | 6.7 (−25.72, | −6.1 (−34.77, |
| (95% CI) [1] | 24.79) | 41.25) | 27.31) |
| Two-sided P-value | 0.1967 | 0.7109 | 0.7233 |
| CD79B mutation | | | |
| CD79B_Mut | 3/4 75.0 | 6/13 46.2 | 9/17 52.9 |
| CD79B_WT | 3/6 50.0 | 6/21 28.6 | 9/27 33.3 |
| Difference in ORR | 25.0 (−37.11, | 17.6 (−15.11, | 19.6 (−10.20, |
| (95% CI) [1] | 69.78) | 48.53) | 46.87) |
| Two-sided P-value | 0.4292 | 0.2972 | 0.1977 |
| CD79B Functional mutation | | | |
| CD79B ITAM_Mut | 2/2 100.0 | 6/13 46.2 | 8/15 53.3 |
| CD79B ITAM_WT | 4/8 50.0 | 6/21 28.6 | 10/29 34.5 |
| Difference in ORR | 50.0 (−28.56, | 17.6 (−15.11, | 18.9 (−11.55, |
| (95% CI) [1] | 79.49) | 48.53) | 46.83) |
| Two-sided P-value | 0.1967 | 0.2972 | 0.2280 |

[1] difference 95% CI were based on Miettinen and Nuriminen method
[2] chi-square test

### Conclusion

Patients with higher expression of PAX5 or mutations of NOTCH1 or CD79B showed better response upon administration of Compound 1 in monotherapy treatment regimens. As such, screening of non-GCB DLBCL patients for PAX5 expression or NOTCH1 or CD79B mutations before treatment is a useful marker in determining patient response to Compound 1 and whether treatment with Compound 1 should be continued.

### Example 4B: Treatment of Non-GCB DLBCL with a BTK Inhibitor in Combination with an Anti-CD20 Antibody

### Methods

Combination therapy schedule 1: Compound 1 was administered orally every day in each cycle at 160 mg BID in 28-day cycles, in combination with obinutuzumab, in patients with R/R non-GCB DLBCL. Obinutuzumab was administered for up to 6 cycles consistent with the U.S. label regimen (100 mg at Day 1 Cycle 1, 1000 mg at Day 8 and Day 15, 900 mg at Day 2 Cycle 1 followed by 1000 mg on day one of cycles 2-6).

Combination therapy schedule 2: Compound 1 was administrated orally every day in each cycle at 160 mg BID in 28-day cycles, in combination with rituximab, in patients with R/R non-GCB DLBCL. Rituximab was administered at 375 mg/m2 intravenously on Cycle 1 Days 1, 8, 15, 22, and on Day 1 of Cycles 4, 6, 8, 10. Compound 1 was administered at least 30 minutes prior to the initiation of the rituximab infusion.

42 patients with non-GCB DLBCL were subjected to Compound 1 plus anti-CD20 antibody Combination therapy schedule 1 or 2.

### Results

The unadjusted ORR in non-GCB DLBCL was 22.7% and 35% for the two combo-therapies. For patients who GEP-confirmed activated B-cell DLBCL classification, the ORR was 50% and 40%, respectively.

The expression of approximately 90 lymphoma-associated genes from the HTG GEP assay were analyzed by R package limma for correlation with response to Combination therapy schedules 1 to 2. For the 12 non-GCB patients with HTG gene expression profiles, PIM1, BCL2, and FOXP1 expression was higher in combination therapy responders. (See FIG. 4).

Patients with non-GCB DLBCL with CD79B mutations (n=8) showed significantly higher ORR than patients without CD79B mutations. (see Table 2).

TABLE 2

| | Compound 1 Combination therapy | | |
|---|---|---|---|
| | Plus obinutuzumab | Plus rituximab | |
| | (N = 22) | (N = 20) | Total (N = 42) |
| non-GCB | Response/Patients ORR | Response/Patients ORR | Response/Patients ORR |
| Overall Response | | | |
| Non-GCB DLBCL Patients | 5/22 22.7 | 7/20 35.0 | 12/42 28.6 |
| Patients with Sequence | 5/16 31.3 | 7/18 38.9 | 12/34 35.3 |
| Available | | | |
| Non-GCB DLBCL Patients | 4/15 26.7 | 7/18 38.9 | 11/33 33.3 |
| with Sequence Available | | | |
| ABC Subtype of | 3/6 50.0 | 2/5 40.0 | 5/11 45.5 |
| DLBCL Patients | | | |
| NOTCH1 | | | |
| NOTCH1_Mut | 2/3 66.7 | 0/2 0.0 | 2/5 40.0 |
| NOTCH1_WT | 2/12 16.7 | 7/16 43.8 | 9/28 32.1 |
| Difference in ORR | 50.0 (−6.15, | −43.8 (−67.39, | 7.9 (−27.98, |
| (95% CI) [1] | 84.24) | 28.69) | 49.62) |
| Two-sided P-value | 0.0798 | 0.2315 | 0.7314 |
| MYD88/CD79B | | | |
| MYD88_L265P/CD79B_Mut | 3/3 100.0 | 0/1 0.0 | 3/4 75.0 |
| Other | 1/12 8.3 | 7/17 41.2 | 8/29 27.6 |
| Difference in ORR | 91.7 (29.61, | −41.2 (−64.57, | 47.4 (−2.08, |
| (95% CI) [1] | 98.58) | 44.19) | 74.68) |
| Two-sided P-value | 0.0013 | 0.4117 | 0.0593 |
| MYD88 mutation | | | |
| MYD88_L265P | 3/3 100.0 | 1/3 33.3 | 4/6 66.7 |
| MYD88_WT | 1/12 8.3 | 6/15 40.0 | 7/27 25.9 |
| Difference in ORR | 91.7 (29.61, | −6.7 (−47.89, | 40.7 (−1.36, |
| (95% CI) [1] | 98.58) | 47.64) | 70.35) |
| Two-sided P-value | 0.0013 | 0.8288 | 0.0555 |
| CD79B mutation | | | |
| CD79B_Mut | 3/3 100.0 | 3/5 60.0 | 6/8 75.0 |
| CD79B_WT | 1/12 8.3 | 4/13 30.8 | 5/25 20.0 |
| Difference in ORR | 91.7 (29.61, | 29.2 (−19.61, | 55.0 (15.75, |
| (95% CI) [1] | 98.58) | 67.42) | 78.76) |
| Two-sided P-value | 0.0013 | 0.2545 | 0.0041 |
| CD79B Functional mutation | | | |
| CD79B ITAM_Mut | 3/3 100.0 | 2/3 66.7 | 5/6 83.3 |
| CD79B ITAM_WT | 1/12 8.3 | 5/15 33.3 | 6/27 22.2 |
| Difference in ORR | 91.7 (29.61, | 33.3 (−22.23, | 61.1 (17.03, |
| (95% CI) [1] | 98.58) | 71.01) | 82.01) |
| Two-sided P-value | 0.0013 | 0.2796 | 0.0041 |

[1] difference 95% CI were based on Miettinen and Nuriminen method
[2] chi-square test

### Conclusion

The combination therapy results were very different from the monotherapy results. The enrichment of PAX5 expression upon administration of the mono-therapy was not noted. Instead, increased expression of PIM41, BCL2, and FOXP1 was seen for the Combination Therapy patients. Patients with CD79B mutations showed significantly better response to combo-therapies. Therefore, screening of non-GCB DLBCL patients for PIM1, BCL2, or FOXP1 expression or CD79B mutation before treatment are useful markers in determining patient response to Compound 1 with an anti-CD20 antibody and whether treatment with the combination should be continued.

### Example 4C: Treatment of Non-GCB DLBCL with BTK Inhibitor Monotherapy, or Combination Therapy

### Methods

Monotherapy schedule 1: Compound 1 was administered orally every day at 160 mg twice daily (BID) in 28-day cycles in patients with R/R non-GCB DLBCL

Monotherapy schedule 2: Compound 1 was administered orally every day in each cycle at 160 mg BID in 28-day cycles in patients with R/R non-GCB DLBCL.

Combination therapy schedule 1: Compound 1 was administered orally every day in each cycle at 160 mg BID in 28-day cycles, in combination with obinutuzumab, in patients with R/R non-GCB DLBCL. Obinutuzumab was administered for up to 6 cycles consistent with the U.S. label regimen (100 mg at Day 1 Cycle 1, 1000 mg at Day 8 and Day 15, 900 mg at Day 2 Cycle 1 followed by 1000 mg on day one of cycles 2-6).

Combination therapy schedule 2: Compound 1 was administrated orally every day in each cycle at 160 mg BID in 28-day cycles, in combination with rituximab, in patients with R/R non-GCB DLBCL. And, rituximab was administered at 375 mg/m2 intravenously on Cycle 1 Days 1, 8, 15, 22, and on Day 1 of Cycles 4, 6, 8, 10. Compound 1 was administered at least 30 minutes prior to the initiation of the rituximab infusion.

A total of 121 patients with non-GCB DLBCL were included for response evaluation. 79 patients were subjected to Compound 1 monotherapies (Monotherapy Schedule 1 or 2), and 42 patients were subjected to Compound 1 plus anti-CD20 antibody Combination therapy (Combination therapy schedule 1 or 2).

56 non-GCB patients were further subtyped by gene expression profiling (GEP) using the HTG EdgeSeq DLBCL Cell of Origin Assay. The expression of approximately 90 lymphoma-associated genes from the HTG GEP assay were analyzed by R package limma for correlation with response to zanubrutinib treatment.

### Results

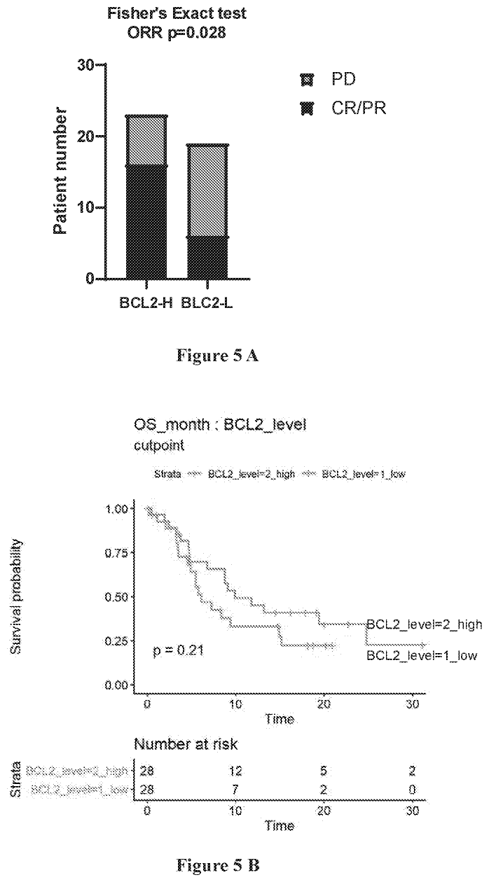

Median expression value of BCL2 was used as threshold for defining BCL2-high and BCL2-low groups. Total 28 BCL2-high (BCL2-H) and 28 BCL2-low (BCL2-L) patients were investigated. Patients high BCL2 expression had significantly better BOR (16/28, 57% vs. 6/28, 21%, p=0.028) and longer PFS (5.3 m vs. 2.8 m, p=0.03)/OS (10 m vs. 6 m, p=0.21). See FIGS. 5A, 5B and 5C.

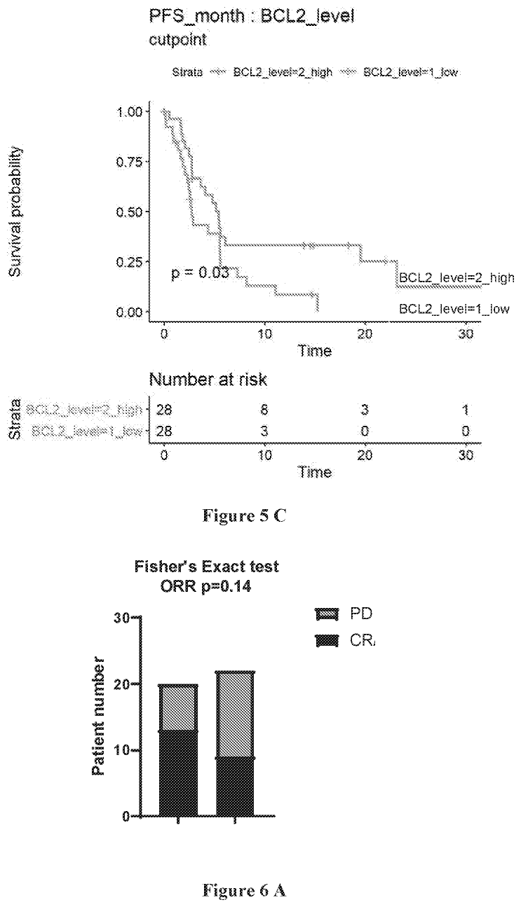

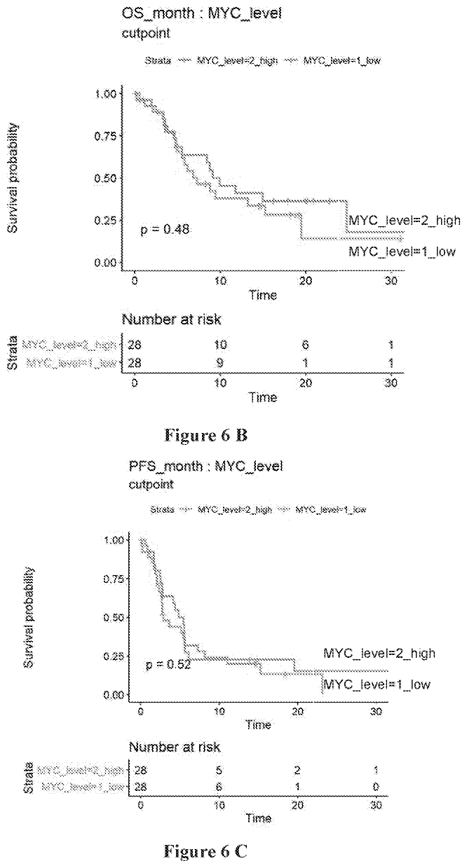

Median expression value of MYC was used as threshold for defining MYC-high and MYC-low groups. Total 28 MYC-high and 28 MYC-low patients were investigated. Patients high MYC expression tended to have better BOR (13/28, 46% vs. 9/28, 32%, p=0.14) and longer PFS (5.4 m vs. 2.9 m, p=0.52)/OS (10 m vs. 7 m, p=0.48). See FIGS. 6A, 6B and 6C.

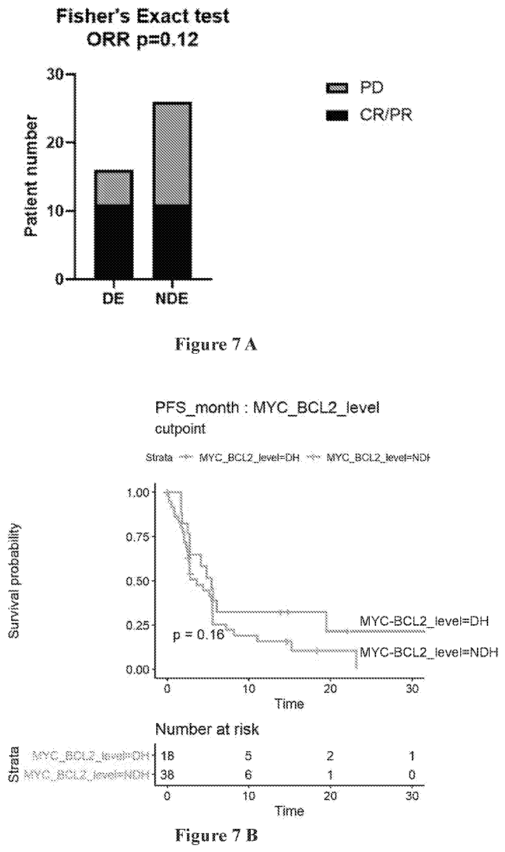

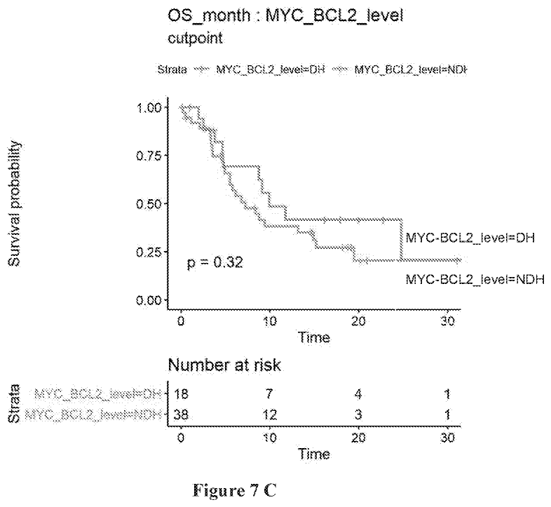

Median expression value of BCL2/MYC were used as threshold for defining double expressor (DE) or non-DE. Patients (18 DE and 38 non-DE) were subjected to the two monotherapies and the two Combination therapies. Patients with MYC and BCL2 double expressor DLBCL tended to have higher ORR (11/18, 61% vs 11/38, 29%; P=0.12) and longer progression-free survival (5.4 months vs 3.6 months; P=0.16) and overall survival (10 months vs 7 months, P=0.32). See FIGS. 7A, 7B, and 7C.

### Conclusion

Patients with MYC, BCL2, or BCL2/MYC double expressor DLBCL tended to have higher ORR and longer progression-free and overall survival with higher response rates to in the four Compound 1 treatment studies. Hence, screening of non-GCB DLBCL patients for MYC, BCL2, or BCL2/MYC double expressor expression after treatment is a useful marker in determining patient response to Compound 1 and Compound 1 with an anti-CD20 antibody, and whether treatment with the monotherapy or combination therapy should be continued.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method of increasing the response of a cancer patient for treatment with a BTK inhibitor, the method comprising:
a) administration of a BTK inhibitor to the patient;
b) assaying for gene expression of PAX5, CD79B mutation or NOTH1 mutation in a cancer sample obtained from the patient;
c) comparing the gene expression of PAX5, CD79B mutation or NOTH1 mutation in the cancer sample obtained from a patient treated with a BTK inhibitor with that of a cancer sample prior to BTK inhibitor administration;
d) wherein increased expression of PAX5, CD79B mutation or NOTH1 mutation indicates that the patient will be sensitive to BTK inhibitor treatment.

2. The method of claim 1, wherein the BTK inhibitor is Compound 1.

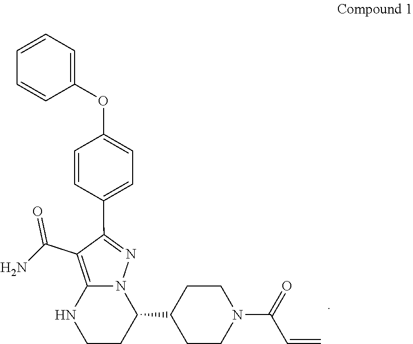 [SMILES: C=CC(=O)N1CCC([C@@H]2CCNc3c(C(N)=O)c(-c4ccc(Oc5ccccc5)cc4)nn32)CC1]

3. The method of claim 2, wherein Compound 1 is administered at 320 mg once daily.

4. The method of claim 2, wherein Compound 1 is administered at 160 mg twice daily.

5. The method of claim 3 or 4 wherein Compound 1 is administered in 28 day cycles.

6. The method of claim 1, wherein the cancer sample is taken from a patient with non-GCB-DLBCL.

7. The method of claim 1, wherein the cancer sample is taken from a patient with ABC-DLBCL.

8. A method of increasing the response of a cancer patient for treatment with a BTK inhibitor in combination with an anti-CD20 antibody, the method comprising:
a) administration of a BTK inhibitor in combination with an anti-CD20 antibody to the patient;
b) assaying for gene expression of any one of PIM1, BCL2, FOXP1, MYC or CD79B mutation in a cancer sample obtained from the patient;
c) comparing the gene expression of any one of PIM1, BCL2, FOXP1, MYC or CD79B mutatiin the cancer sample obtained from a patient treated with a BTK inhibitor in combination with an anti-CD20 antibody with that of a cancer sample prior to BTK inhibitor in combination with an anti-CD20 antibody administration;
d) wherein increased expression of PIM1, BCL2, FOXP1, MYC or CD79B mutation indicates that the patient will be sensitive to BTK inhibitor treatment.

9. The method of claim 8, wherein the BTK inhibitor is Compound 1.

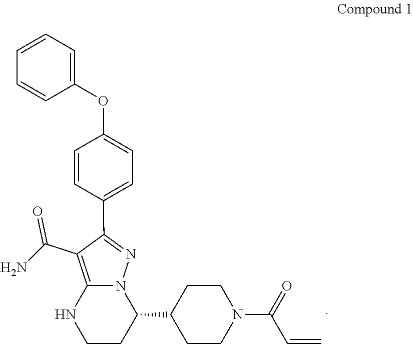 [SMILES: C=CC(=O)N1CCC([C@@H]2CCNc3c(C(N)=O)c(-c4ccc(Oc5ccccc5)cc4)nn32)CC1]

10. The method of claim 9, wherein Compound 1 is administered at 320 mg once daily.

11. The method of claim 9, wherein Compound 1 is administered at 160 mg twice daily.

12. The method of claim 10 or 11 wherein Compound 1 is administered in 28 day cycles.

13. The method of claim 8, wherein the anti-CD20 antibody is rituximab, ibritumomab tiuxetan, tositumomab, ofatumumab or obinutuzumab.

14. The method of claim 13, wherein the anti-CD20 antibody is rituximab.

15. The method of claim 13, wherein the anti-CD20 antibody is obinutuzumab.

16. The method of claim 8, wherein expression of PIM1 and BCL2 is increased.

17. The method of claim 8, wherein expression of PIM1, BCL2 and FOXP1 is increased.

18. The method of claim 8, wherein expression of PIM1, BCL2, FOXP1 and MYC is increased.

19. The method of claim 8, wherein the cancer sample is taken from a patient with non-GCB-DLBCL.

20. The method of claim 8, wherein the cancer sample is taken from a patient with ABC-DLBCL.
